(19) United States
(12) Patent Application Publication
(10) Pub. No.: US 2006/0140899 A1
(43) Pub. Date: Jun. 29, 2006

(54) Skin cleansing system comprising an anti-adherent formulation and a cationic compound

(75) Inventors: David W. Koenig, Menasha, WI (US); Karyn Clare Schroeder, Neenah, WI (US); Candace Dyan Krautkramer, Neenah, WI (US); Jason C. Cohen, Appleton, WI (US); Douglas Robert Hoffman, Oshkosh, WI (US); Katherine D. Stahl, Appleton, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc., Neenah, WI (US)

(21) Appl. No.: 11/024,266

(22) Filed: Dec. 28, 2004

(51) Int. Cl. A61K 8/49; A61K 8/97; C11D 1/62

(52) U.S. Cl. 424/70.27; 424/70.28; 424/74; 424/443

(57) ABSTRACT

Cleansing systems and methods are disclosed for improving the cleaning of skin. The cleansing systems comprise a first product comprising an anti-adherent formulation and a second product comprising a cationic compound capable of binding contaminants located on the skin. The first product is wiped across the skin of a user to introduce a film of the anti-adherent formulation onto the surface of the skin. This film reduces the amount of contaminant that is retained on the skin. The second product is wiped across the skin of a user to further dislodge and remove contaminants from the skin. The cleansing systems result in cleaner, healthier skin.

### BACKGROUND OF INVENTION

The present invention generally relates to cleansing systems for improving skin health. More particularly, the present invention relates to a cleansing system comprising a first product comprising an anti-adherent formulation and a second product comprising a soil attractive system. The cleansing systems are highly effective in binding and removing from the surface of skin a broad range of microorganisms and other contaminants such as fungi, yeasts, molds, protozoa, and viruses. In one embodiment, the first product is wiped across the surface of the skin to introduce an anti-adherent film that can keep contaminants from adhering to the skin's surface. The second product comprises a cationic compound capable of binding to the contaminants and holding them in the second product away from the skin.

Human skin is exposed to various contaminants everyday through both contact with various biological fluids such as urine and feces as well as contact with numerous environmental factors. Examples of contaminants that the skin contacts everyday include yeast, fungi, mold, protozoa, and viruses. Although most microbes are negatively charged due to their chemistry and structures, they can adhere to skin, which is also typically negatively charged, through various interactions such as electrostatic interactions, hydrophobic interactions, and ligand interactions. Although these attachment mechanisms are not completely understood, their cumulative effect can tightly bind numerous microbes such as Candida albicans to skin resulting in inflammation and irritation. Furthermore, these contaminants can initiate an elaborate cascade of immunological events upon contact with viable skin cells leading to further skin irritation, inflammation, and even infection. Skin cleansing on a daily basis can prevent or minimize skin irritation and inflammation caused by these contaminants.

Conventionally, cleaning of the skin has included any activity that kills, binds, and/or removes contaminants present on the skin's surface. Microbiocidal agents contained in many cleaning products may, however, irritate the skin of some users due to the potentially harsh chemicals utilized to provide the antimicrobial effect. Additionally, some products utilized, such as wet wipes, contain surfactants and/or alcohol or other additives that, while effective against numerous microbes, may dry out or chafe skin. As such, although products are generally effective in cleaning and maintaining healthy skin, some products may be unsuitable for use by some people.

Additionally, properly cleaning skin in regions such as in the perianal, uro-genital, and vaginal regions may be difficult due to the topography of the skin and the presence of hair follicles. For example, a common problem encountered by many individuals during cleanup after a bowel movement is the occasional sticking of fecal material to the skin in the perianal and related regions. This sticking can result in a number of undesirable situations including, for example, transfer of the fecal material to undergarments and unwanted odors. Additionally, because fecal material generally contains bacteria and active enzymes, the presence of this material in the anal region after bowel movement cleanup can also result in skin irritation, redness, and even inflammation and infection for sensitive individuals.

Based on the foregoing, it is clear that maintaining clean and healthy skin is difficult, yet important. As such, there is a need in the art for cleansing systems and products that can improve cleaning of the skin without irritating or chafing the skin of the user. It would also be desirable for the cleansing systems or products to simply remove microbes and contaminants from the skin surface without killing the microbe so as to avoid further infection.

### SUMMARY OF THE INVENTION

The present invention is directed to novel cleansing systems that contain one or more products comprising an anti-adherent formulation in combination with a cationic compound. Generally, the cleansing system comprises a first product comprising an anti-adherent formulation and a second product comprising a cationic compound capable of binding contaminants located on or near the skin. The first product of the cleansing system is wiped or rubbed across a region of skin to impart an anti-adherent formulation onto the region such that a reduced amount of contaminant is retained on the skin in the region and surrounding areas. Additionally, the contaminants are then bound to a cationic compound contained in a second product. This results in further removing contaminants from the skin's surface, which can result in cleaner, healthier skin.

The present invention is also directed to methods of using the cleansing system for cleaning skin. These methods comprise contacting the skin with a first product comprising an anti-adherent formulation and then contacting the skin with the second product comprising a cationic compound. This contacting introduces the anti-adherent formulation onto the skin, reducing the amount of contaminants that adhere to the skin; and further, binds contaminants, pulling the contaminants into the second product and away from the skin's surface. Because a reduced amount of contaminants remains on the surface of the skin, the skin is cleaner and healthier.

As such, the present invention is directed to a cleansing system for improving skin health. The cleansing system comprises a first product comprising an anti-adherent formulation; and a second product comprising a cationic compound capable of binding contaminants located on the skin. The cationic compound has an effective charge density of from about 0.1 microequivalents/g to about 8000 microequivalents/g. The second product has an effective charge density of at least about 2000 microequivalents/100 g.

The present invention is further directed to a product for improving skin health. The product comprises an anti-adherent formulation, and a cationic compound capable of binding contaminants located on the skin. The cationic compound has an effective charge density of from about 0.1 microequivalents/g to about 8000 microequivalents/g. The product has an effective charge density of at least about 2000 microequivalents/100 g.

The present invention is further directed to a process for improving skin cleansing. The process comprises contacting the skin with a first product comprising an anti-adherent formulation and contacting the skin with a second product comprising a cationic compound capable of binding contaminants located on the skin. The cationic compound has an effective charge density of from about 0.1 microequivalents/g to about 8000 microequivalents/g. The second product has an effective charge density of at least about 2000 microequivalents/100 g.

Other features of the present invention will be in part apparent and in part pointed out hereinafter.

### DETAILED DESCRIPTION OF THE PREFERRED EMBODIMENT

The present invention is generally directed to cleansing systems and methods for assisting in the cleaning of skin. Specifically, the present invention relates to a cleansing system comprising one or more products. In one embodiment, the cleansing system comprises a first product and a second product. When utilized to clean the skin, the first product transfers an anti-adherent formulation onto the skin, minimizing the amount of contaminants that remains on the skin after contact with the contaminants. The presence of an anti-adherent formulation on the skin may additionally make the removal of contaminants easier. The second product comprises a cationic compound capable of binding the contaminants and pulling the contaminants away from the surface of the skin and into the product. When used in combination, this cleansing system results in cleaner, healthier skin. Alternatively, the system may comprise a single product comprising both the anti-adherent formulation and the cationic compound.

As noted above, the cleansing systems described herein comprise one or more products. Typically, the cleansing system comprises a first product and a second product. In one embodiment, products suitable for use in the cleansing systems of the present invention are pharmaceutically-acceptable and compatible carrier materials. Carrier materials suitable for use in the instant invention include those well-known for use in the cosmetic and medical arts as a basis for ointments, lotions, creams, salves, aerosols, suppositories, gels, foams, washes, mists and sprays, and the like.

In another embodiment, the products for use in the present invention comprise a base substrate. Base substrates suitable for use in the products of the present invention can be made from various materials and fibers, and are desirably soft to the touch. Optionally, the products described herein may be flushable after use. The products may be dry or wet to the touch, and may feel like conventional bath tissue, or like a wet wipe. The base substrate can be made from pulp fibers, other natural fibers, cellulose fibers, synthetic fibers such as polypropylene or polylactic acid, and the like. The base substrate may be woven or nonwoven and may be sized for easy single handed use. Although size is not critical, a suitable size may be, for example, 6 inches by about 4 inches.

One desirable base substrate is a tissue product substrate. The present invention is useful with tissue products and tissue paper in general, including but not limited to conventionally felt-pressed tissue paper, high bulk pattern densified tissue paper, and high bulk, uncompacted tissue paper. The tissue paper can be of a homogenous or multi-layered construction, and tissue paper products made therefrom can be of a single-ply or multi-ply construction. The tissue paper desirably has a basis weight of between about 10 g/m² and about 65 g/m², and a density of about 0.6 g/cc or less. More desirably, the basis weight will be about 40 g/m² or less and the density will be about 0.3 g/cc or less. Most desirably, the density will be between about 0.04 g/cc and about 0.2 g/cc. Unless otherwise specified, all amounts and weights relative to the paper are on a dry basis. Stretch in the machine direction can be in the range of from about 5% to about 20%. Stretch in the cross-machine direction can be in the range of from about 3% to about 20%. Tensile strengths in the machine direction can be in the range of from about 100 to about 5,000 grams per inch of width. Tensile strengths in the cross-machine direction are in the range of from about 50 grams to about 2,500 grams per inch of width. Absorbency is typically from about 5 grams of water per gram of fiber to about 9 grams of water per gram of fiber.

Conventionally pressed tissue paper and methods for making such paper are well known in the art. For example, high bulk pattern densified tissue paper suitable for use in the present invention is disclosed in U.S. Pat. No. 3,301,746 (Sanford et al.), issued Jan. 31, 1967; U.S. Pat. No. 3,974,025 (Ayers), issued Aug. 10, 1976; and U.S. Pat. No. 4,191,609 (Trokhan), issued Mar. 4, 1980; and U.S. Pat. No. 4,637,859 (Trokhan), issued Jan. 20, 1987; all of which are incorporated by reference. Additionally, uncompacted, nonpattern-densified tissue paper structures suitable for use in the present invention are described in U.S. Pat. No. 3,812,000 (Salvucci et al.), issued May 21, 1974 and U.S. Pat. No. 4,208,459 (Becker et al.), issued Jun. 17, 1980, both of which are incorporated by reference.

Such paper is typically made by depositing a papermaking furnish on a foraminous forming wire, often referred to in the art as a Fourdrinier wire. Once the furnish is deposited on the forming wire, it is referred to as a web. The web is dewatered by pressing the web and drying at an elevated temperature. The particular techniques and typical equipment for making webs according to the process just described are well known to those skilled in the art. In a typical process, a low consistency pulp furnish is provided from a pressurized headbox, which has an opening for delivering a thin deposit of pulp furnish onto the Fourdrinier wire to form a wet web. The web is then typically dewatered to a fiber consistency of between about 7% and about 25% (total web weight basis) by vacuum dewatering and further dried by pressing operations wherein the web is subjected to pressure developed by opposing mechanical members, for example, cylindrical rolls. The dewatered web is then further pressed and dried by a steam drum apparatus known in the art as a Yankee dryer. Pressure can be developed at the Yankee dryer by mechanical means such as an opposing cylindrical drum pressing against the web. Multiple Yankee dryer drums can be employed, whereby additional pressing is optionally incurred between the drums. The formed sheets are considered to be compacted since the entire web is subjected to substantial mechanical compressional forces while the fibers are moist and are then dried while in a compressed state.

The papermaking fibers utilized in preparing tissue paper for the products of the present invention will normally include fibers derived from wood pulp. Other cellulosic fibrous pulp fibers, such as cotton linters, bagasse, etc., can be utilized and are intended to be within the scope of this invention. Synthetic fibers, such as rayon, polyethylene, polypropylene, and bicomponent fibers of polypropylene with polyester or polyethylene fibers, can also be utilized in combination with natural cellulosic fibers. One exemplary polyethylene fiber that can be utilized is Pulpex.RTM., available from Hercules, Inc. (Wilmington, Del.).

Applicable wood pulps include chemical pulps, such as Kraft, sulfite, and sulfate pulps, as well as mechanical pulps including, for example, groundwood, thermo-mechanical pulp and chemically modified thermo-mechanical pulp. Chemical pulps, however, are typically desirable since they impart a superior tactile sense of softness to tissue sheets made therefrom. Pulps derived from both deciduous trees and coniferous trees can be utilized. Also useful in the present invention are fibers derived from recycled paper, which can contain any or all of the above categories as well as other non-fibrous materials such as fillers and adhesives used to facilitate the original papermaking.

In addition to papermaking fibers, the papermaking furnish used to make tissue paper structures can have other components or materials added thereto as can be or later become known in the art. The types of additives desirable will be dependent upon the particular end use of the tissue sheet contemplated. For example, in products such as bath tissue, paper towels, facial tissues and other similar products, adequate wet strength is a desirable attribute. Thus, it is often desirable to add to the papermaking furnish chemical substances known in the art as “wet strength” additives.

In addition to wet strength additives, it can also be desirable to include in the papermaking fibers certain dry strength and lint control additives known in the art. In this regard, starch binders have been found to be particularly suitable. In addition to reducing tinting of the finished tissue paper product, low levels of starch binders also impart a modest improvement in the dry tensile strength without imparting stiffness that could result from the addition of high levels of starch. Typically, the starch binder is included in an amount such that it is retained at a level of from about 0.01 to about 2%, preferably from about 0.1 to about 1%, by weight of the dry tissue paper.

Another desirable base substrate for the products of the present invention is an absorbent substrate, such as for use in absorbent products. The present invention is primarily described herein in combination with an absorbent substrate for use in an absorbent disposable diaper. It is readily apparent to one skilled in the art based on the disclosure herein, however, that the anti-adherent formulations and cationic compounds described herein can also be used in combination with numerous other disposable absorbent articles having absorbent substrates such as, for example, training pants, adult incontinence garments and pads, feminine napkins, hand towels, wound dressings, and the like.

Generally, the diaper includes a substantially liquid impermeable outer cover, a porous, liquid permeable bodyside liner positioned in facing relation with the outer cover, and an absorbent substrate, such as an absorbent pad, which is located between the outer cover and the bodyside liner.

Generally, the absorbent substrate of a diaper suitable for use in the present invention may comprise a matrix of hydrophilic fibers, such as a web of cellulosic fluff, mixed with particles of a high-absorbency material commonly known as superabsorbent material. In a particular embodiment, the absorbent substrate comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be non-uniformly mixed. The fluff and superabsorbent particles may also be selectively placed into desired zones of the absorbent substrate to better contain and absorb body exudates. The concentration of the superabsorbent particles may also vary through the thickness of the absorbent substrate. Alternatively, the absorbent substrate may comprise a laminate of fibrous webs and superabsorbent material or other suitable means of maintaining a superabsorbent material in a localized area.

The absorbent substrate may have any of a number of shapes. For example, the absorbent substrate may be rectangular, I-shaped, or T-shaped. It is generally preferred that the absorbent substrate be narrower in the crotch area than in the front or rear portions of the diaper. The size and the absorbent capacity of the absorbent substrate should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent substrate include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from BASF Corporation located in Florham Park, N.J., and DOW DRYTECH 2035LD polymer available from Dow Chemical Company located in Midland, Mich.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent substrate in an amount of from about 5 to about 90 weight percent based on a total weight of the absorbent substrate.

Processes for the manufacture of diapers suitable for use in connection with the instant application and other diaper components suitable for use on the diaper are described in U.S. Pat. No. 4,798,603 issued Jan. 17, 1989 to Meyer et al.; U.S. Pat. No. 5,176,668 issued Jan. 5, 1993, to Bernardin; U.S. Pat. No. 5,176,672 issued Jan. 5, 1993 to Bruemmer et al.; U.S. Pat. No. 5,192,606 issued Mar. 9, 1993 to Proxmire et al.; and U.S. Pat. No. 5,509,915 issued Apr. 23, 1996 to Hanson et al., the disclosures of which are hereby incorporated by reference.

As stated above, the cleansing systems of the present invention comprise one or more products described above for use alone or in combination. Generally, the products comprise an anti-adherent formulation and/or a cationic compound capable of binding contaminants located on the skin. By way of example, in one embodiment, the cleansing system comprises a first product as described above comprising an anti-adherent formulation and a second product as described above comprising a cationic compound capable of binding contaminants located on the skin. In another embodiment, the cleansing system comprises one product comprising both an anti-adherent formulation and a cationic compound capable of binding contaminants located on the skin.

When one product comprises the combination of an anti-adherent formulation and a cationic compound capable of binding to contaminants located on the skin, the formulation or compound can suitably be applied in a pattern on the base substrate. Suitably, the formulation or compound is applied in a striped or checkered pattern. For example, in one embodiment, a striped pattern can be applied through striped gravure printing of the anti-adherent formulation and/or the cationic compound onto the surface of the base substrate.

The anti-adherent formulation may be hydrophobic or hydrophilic. This formulation is transferred to the skin of a user when a product described above is contacted with the skin of the user by wiping or rubbing the product across the skin or by wearing a product comprising the anti-adherent formulation. The formulation forms a film on the skin, which may result in a reduced amount of contaminants adhering to the skin. Additionally, the film may result in easier removal of any contaminants that do remain on the skin. This can result in cleaner, healthier skin.

In one embodiment, the anti-adherent formulation comprises a hydrophobic anti-adherent formulation. In this embodiment, the formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

An emollient is an active ingredient in a formulation that typically softens, soothes, supples, coats, lubricates and/or moisturizes the skin. Generally, emollients accomplish several of these objectives simultaneously. Typically, emollients suitable for use in the anti-adherent formulations described herein are fluids at room temperature such that they impart a soft, lubricious lotion-like feel upon use. Suitable emollients for use in the formulations of the present invention are typically substantially water free. Although the emollient component may contain trace amounts of water as a contaminant without substantially harming the formulation, it is preferred that the amount of water be less than about 5% by weight of the emollient component of the formulation to reduce the likelihood of microbial growth and product destruction.

Suitable emollients for inclusion in the formulations described herein include petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof. Petrolatum and mineral oil are preferred emollients.

The structuring agent utilized in the anti-adherent formulations described herein helps to immobilize the emollient and other components in or on the product. Because some emollients are fluids at room temperature, they may tend to flow or migrate out of or away from the product. The structuring agent reduces the ability of the emollient (and other components) to migrate and keeps the emollient primarily in or on the product.

Suitable structuring agents include animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

The rheology modifier utilized in the anti-adherent formulation increases the melt point viscosity of the formulation so that the formulation readily remains in or on the product and does not substantially migrate into or out of the product, while substantially not affecting the transfer of the anti-adherent formulation to the skin. Additionally, the rheology modifier helps the anti-adherent formulation to maintain a high viscosity at elevated temperatures, such as those encountered during storage and transportation.

Suitable rheology modifiers include combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, and silicas.

The anti-adherent compound included in the anti-adherent formulations described herein acts to prevent the adherence of contaminants to the skin during and after contact with the contaminant. The presence of the anti-adherent compound in the formulation results in a decreased amount of contaminants that adhere to the skin. Without being bound to a particular theory, it is believed that the anti-adherent compound attaches to the skin through electrical interaction with the skin and remains tightly bound thereto after deposit. When contamination occurs, microbes, enzymes, and other contaminants, which also typically attach to skin through electrical interactions, are not able to make the attachment to the skin as many of the binding sites are already occupied with anti-adherent compound. Because electrical interaction with the microbes and enzymes and the skin is reduced, much less contaminant remains attached to the skin.

Suitable anti-adherent compounds include alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

When utilized in a product comprising a base substrate, the hydrophobic anti-adherent formulation described above may have specific melt point and process temperature viscosities, as defined herein. These viscosities are important for at least two reasons. First, the higher the melt point or process temperature viscosity, the less likely the anti-adherent formulation is to penetrate into the inner surface of the base substrate. The less formulation that is able to penetrate into the interior of the base substrate, results in more formulation on the surface of the base substrate that can transfer to the user's skin. Secondly, the higher the viscosity of the formulation at or above the melting point of the formulation, the less likely the formulation will be to migrate at typical or adverse storage or temperature conditions.

The hydrophobic anti-adherent formulations described above have a melt point viscosity of from about 5000 cPs to about 1,000,000 cPs, desirably from about 50,000 cPs to about 800,000 cPs, and more desirably from about 100,000 cPs to about 500,000 cPs. As used herein, the term “melt point viscosity” means the viscosity of the formulation at the point in time when the formulation visually becomes a liquid. Formulations having melt point viscosities in these ranges significantly improve the ability of the formulation to remain on the surface of the base substrate and the formulation maintains a high viscosity at elevated temperatures, such as those encountered during storage and shipment.

Additionally, to improve application to the surface of the base substrate, the hydrophobic formulations described herein have a process temperature viscosity of from about 50 cPs to about 50,000 cPs, desirably from about 75 cPs to about 10,000 cPs, and more desirably from about 100 cPs to about 5,000 cPs. The process temperature is typically from about 5° C. to about 10° C. above the melting point of the lotion formulation.

In an alternative embodiment of the present invention, the anti-adherent formulation may comprise a hydrophilic formulation. The hydrophilic formulation may comprise from about 30% (by weight) to about 79.98% (by weight) glycol, from about 10% (by weight) to about 58% (by weight) polyethylene glycol having a melting point greater than about 35° C., from about 10% (by weight) to about 58% (by weight) fatty acid or fatty alcohol, from about 0.01% (by weight) to about 10% (by weight) dimethicone or dimethiconol, and from about 0.01% (by weight) to about 1% (by weight) anti-adherent compound.

The glycol component of the hydrophilic anti-adherent formulation acts to ensure a high degree of compatibility between the components and ensures that a homogeneous formulation is produced. Suitable glycols include, for example, propylene glycol, butylene glycol, 1,3 butylene glycol, polyethylene glycols that are liquid at room temperature, dipropylene glycol, methylpropane glycol, silicone glycol, polypropylene glycol, hydrogenate starch hydrolysates, and combinations thereof. Polyethylene glycols that are liquid at room temperature include low molecular weight polyethylene glycols, such as those having a molecular weight of less than about 720 (e.g., PEG 600).

Polyethylene glycols having a melting point greater than about 35° C. are included in the anti-adherent formulation as structurants. Suitable polyethylene glycols in this category include polyethylene glycols having a molecular weight greater than about 720.

The fatty acid or fatty alcohol is included in the anti-adherent formulation as structurants and emollients. Suitable fatty acids or fatty alcohols include those having a carbon chain length of from about 14 to about 22 carbon atoms. Specific examples include myristyl alcohol, cetyl alcohol, stearyl alcohol, and behenyl alcohol.

The dimethicone or dimethiconol is included in the formulation as an emollient. Suitable examples include, for example, Dow Corning 200 and Dow Corning 1503.

In another embodiment of the present invention, the anti-adherent formulation may comprise from about 99% (by weight) to about 99.99% (by weight) fatty acid ester having a melting point greater than 35° C. and from about 0.01% (by weight) to about 1% (by weight) anti-adherent compound. The fatty acid ester is a structurant and an emollient and may include compounds such as, for example, myristal myristate, cetyl palmitate, cetyl benzoate, cetyl lactate, steryl behenate, and combinations thereof.

In another embodiment of the present invention, the anti-adherent formulation may comprise from about 99% (by weight) to about 99.99% (by weight) Dow Corning 7-3076 polyamide blend and from about 0.01% (by weight) to about 1% (by weight) anti-adherent compound. The Dow Corning 7-3076 is a proprietary blend of Nylon 6111 dimethicone copolymer and PPG 3 myristal ether.

In still another embodiment of the present invention, the anti-adherent formulation may comprise one or more of the following components: petrolatum, glycerin, mineral oil, and olive oil.

The products described herein contain an amount of anti-adherent formulation such that, upon wiping across the surface of the skin, an effective amount of formulation is transferred to the skin surface. Specifically, when the product is a pharmaceutically acceptable carrier, the product typically contains from about 0.01% (wt/vol) to about 5% (wt/vol) of anti-adherent formulation (based on the total weight of the product). More suitably, the product contains from about 0.01% (wt/vol) to about 1% (wt/vol) of anti-adherent formulation (based on the total weight of the product).

When the product comprises a base substrate, the product may suitably contain from about 1% (by weight of the base substrate) to about 25% (by weight of the base substrate), desirably from about 1% (by weight of the base substrate) to about 10% (by weight of the base substrate). Based on the disclosure herein, one skilled in the art will recognize that various amounts of anti-adherent formulation may be suitable for different end products.

In addition to the anti-adherent formulation described above, products for use in the cleansing systems of the present invention can also comprise cationic compounds. As used herein, the term “cationic compound” means any compound or ingredient that increases the overall cationic charge of the products used in the cleansing systems of the present invention.

The cationic compounds of the present invention do not necessarily kill or inhibit the growth of microbes, but displace and bind the predominantly negatively charged microbes or other contaminants from the skin surface through positive-negative or negative-positive electrostatic interactions. This is highly advantageous in that the cleansing systems of the present invention do not require an antimicrobial agent to be highly effective. When the cleansing systems of the present invention are utilized on the surface of the skin, microbes are not simply killed and left on the skin, but are actually bound to the cationic compounds and removed from the skin. This may reduce the chance of infection. Further, the cationic compounds used in the products of the present invention are substantially non-toxic and non-irritating to the skin.

Without being bound to a particular theory, it appears that by increasing the attractive forces between the product containing the cationic compounds and the contaminant of the skin's surface in excess of the forces attracting the contaminant to the skin, cleaning of the skin can be significantly enhanced by dislodging and binding the contaminant to the cationic species added to the product. It appears that the cationic compounds interact with the overall net negative charge of the contaminant causing the detachment of the contaminant from the skin through an electrostatic interaction. The interaction between the cationic compounds and the skin appears to be stronger than the combined forces of adhesion that retain the contaminant on the skin including hydrophobic interactions, electrostatic interactions, and ligand interactions. Because the contaminant is released from the skin and bound to the charge modified product, it may be easily and efficiently carried away by the product. This is highly advantageous over more traditional cleansing formulations as the contaminant is not merely dislodged from the skin surface, but is dislodged and then removed from the skin's surface through interactions with the product containing the cationic compounds. A suitable amount of cationic compounds must be added to the products of the present invention such that the forces binding the contaminant to the skin surface, such as hydrophobic interactions, electrostatic interactions, and ligand interactions, can be overcome by the attraction to the cationic species.

In accordance with the present invention, an amount of cationic compounds in excess of the amounts typically used in the manufacturing processes of products suitable for use in the cleansing systems of the present invention is added to the products during manufacturing to alter the electric charge of the product from negative to positive (or from very slightly positive to more positive) to increase the overall effective charge density of the finished product such that the product retains a strongly positive overall effective charge density that is highly effective in binding and removing contaminants from the skin's surface through electrostatic interactions. A suitable amount of cationic compound is added to produce a finished product having an effective cationic charge density of at least about 2000 microequivalents/100 g, more suitably at least about 3000 microequivalents/100 g, and even more suitably at least about 3500 microequivalents/100 g, or more. By significantly increasing the net cationic charge of the finished product, the product can effectively bind and remove contaminants from the skin's surface.

Examples of suitable cationic compounds that can be utilized to increase the overall effective cationic charge density of the cleansing products of the present invention include, for example, polyquaternary amines, such as those sold under the tradename Bufloc 535 (Buckman Laboratories International, Memphis, Tenn.), Nalco 7607 (ONDEO NALCO Company, Naperville, Ill.), Reten 201 (Hercules Inc., Wilmington Del.), Cypro 515 (CIBA Speciality Chemicals, Suffolk Va.), Bufloc 5554 (Buckman Laboratories International, Memphis, Tenn.), and Busperse 5030 (Buckman Laboratories International, Memphis, Tenn.) and cationic polymers, inorganic cationic species, biological cationic polymers, modified chitosan, octadecyldimethyltrimethoxylsil-propylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-oleyl-3-oleyl-amidoethyl imidazoline methylsulfate, 1-ethyl-2-oleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicon-2, and mixtures and combinations thereof. Especially preferred compounds include quaternary compounds, polyelectrolytes, octadecyldimethoxylsilpropylammonium chloride, 1-methyl-2-oleyl-3-oleyl-amidoethyl imidazoline methylsulfate, and 1-ethyl-2-oleyl-3-oleyl-amidoethyl imidazoline ethylsulfate. It would be recognized by one skilled in the art that other cationic compounds commonly known in the art could also be utilized in accordance with the present invention to significantly increase the overall cationic effective charge density of the resulting product.

The cationic compounds for incorporation into the products used in the cleansing systems of the present invention have a net cationic charge, and may sometimes be referred to as anionic exchangers. Typically, the products of the present invention contain cationic compounds having sufficient positive charge to impart improved cleaning characteristics through electrostatic interactions with contaminants and skin. The amount of “cationic charge” on a particular compound can vary substantially and can be measured utilizing several different units. Ion exchangers are sometimes referred to as having a “capacity” which may be measured in microequivalents per gram or milliequivalents per gram, or may be measured in terms of the amount of a certain compound or protein that the ion exchanger will bind. Still another way of referring to the amount of ionic charge that can be bound by the anionic exchanger is in terms of micro or milli-equivalents per unit area. One skilled in the art will recognize that the exchanger capacity units or ionic units can be converted from one form to another to calculate proper amounts of anionic exchanger for use in the present invention.

In accordance with the present invention, the chemical additives utilized to increase the overall effective cationic charge density of the resulting product have a cationic charge. Cationic compounds useful in the present invention typically have an effective charge density of from about 0.1 microequivalents/g to about 8000 microequivalents/g, more preferably from about 100 microequivalents/g to about 8000 microequivalents/g, still more preferably from about 500 microequivalents/g to about 8000 microequivalents/g, and most preferably from about 1000 microequivalents/g to about 8000 microequivalents/g. Although effective charge densities of more than about 8000 microequivalents/g can be used in the cleansing systems of the present invention, such a large charge density is not typically required to realize the benefit of the present invention, and may result in the deterioration of product properties.

As the effective charge density of the cationic material increases, the amount of cationic material required to be added to a product typically decreases. Generally, from about 0.01% (by weight of the product) to about 25% (by weight of the product), preferably from about 0.01% (by weight of the product) to about 10% (by weight of the product) of cationic compound material having the above-described effective charge density will be sufficient to increase the overall cationic charge of the resulting product sufficiently for purposes of the present invention.

The actual amount of cationic material required for introduction into the products of the present invention may be influenced by numerous other factors including, for example, the amount of steric hindrance in the product due to other additives present in the product, accessibility of the charges on the product, competitive reactions by cationic materials for anionic sites, the potential for multilayer adsorption into a product comprising a base substrate, and the potential for precipitation of anionic materials out of solution.

When the products for use in the cleansing systems of the present invention comprise base substrates, the products can further comprise a bodily exudate modifying agent in combination with the anti-adherent formulation and/or cationic compounds described above. As used herein, the term “bodily exudate” means secretions from the human body that have a viscosity greater than that of urine. Bodily exudates include, for example, solid, semi-solid and liquid bowel movements, menses, and other vaginal and anal secretions. Suitably, the bodily exudate modifying agents are capable of reducing the viscosity of bodily exudates, such as feces or menses, upon contact therewith. By reducing the viscosity of the exudates, there may be improved absorption of the exudates into the base substrate of a product and away from the skin of the wearer. This results in improved skin health as numerous compounds contained in the bodily exudates that could damage the skin upon contact therewith are substantially removed from the area of the product in contact with the surface of the skin.

As used herein, the term “bodily exudate modifying agent” refers to a chemical composition capable of reducing the viscosity of bodily exudates, such as feces and menses, through chemical modification to allow for enhanced absorption of the bodily exudates by one or more areas of a product suitable for use in the cleansing system. As such, these bodily exudate modifying agents may be used to enhance the ease of bodily exudate penetration into the base substrate of a product and away from the skin. Suitably, the bodily exudate modifying agent reduces the viscosity of at least some of the bodily exudate by at least about 5%, more suitably, at least about 25%. Such a reduction in viscosity results in the exudates flowing much more easily into a desired area of a product, which may be, for example, the base substrate.

Typically, the bodily exudate modifying agent is present in the product in an amount of from about 0.01% (by weight of the material) to about 30% (by weight of the material). More suitably, the bodily exudate modifying agent is present in the product in an amount of from about 5.0% (by weight of the material) to about 20% (by weight of the material). Based on the disclosure herein, one skilled in the art will recognize that the exact amount of modifying agent required to provide the intended function may vary depending upon the desired application, and the exact location of the modifying agent in the product.

Typically, the bodily exudate modifying agents suitable for use in the present invention can include enzymes, reducing agents, metal-based modifying agents, pore-forming toxins, nanoemulsions, surfactants, and combinations thereof. Enzymes are complex proteins that are produced by cells and act as catalysts in biochemical reactions. More particularly, specific enzymes can be utilized in the present invention to reduce the viscosity of bodily exudates by catalyzing reactions on the surface of the exudates that result in a breakdown of the exudate. Suitable enzymes for use as bodily exudate modifying agents in the products as described herein include amylase, lysozyme, zymolyase, celulase, protease, lipase, urease, elastase, carbohydrase, cathepsin G, myeloperoxidase, cytolysins, such as phospholipase and listeriolysin, streptolysin, perfringolysin, and combinations thereof. Suitable proteases include serine proteases, cysteine proteases, and metalloproteases.

In another embodiment, the bodily exudate modifying agent is a reducing agent. For example, agents that reduce disulfide bonds (—S—S-bonds), as found in colonic mucus (colonic mucus generally comprises various macromolecular glycoproteins linked by disulfide bonds), can effect a significant viscosity reduction in feces having high mucus content, such as runny feces. This reduction of disulfide bonds denatures the various glycoproteins. Without being bound to a particular theory, it is believed that the denaturing of the proteins by the reduction of the mucin disulfide bonds (which function as crosslinks between mucin polymer chains) significantly reduces the average molecular weight of the glycoprotein structure in feces, such as runny feces, to a level well below the “gel point” of the mucin (i.e., long-distance structure becomes impossible due to the relatively small size of the glycoproteins). This reduction in average molecular weight results in a decrease in viscosity.

Suitable reducing agents can include sulfites such as sodium hydrogensulphite, sodium sulfite and sodium dithionite, thiols, thiol alcohols (e.g., 2-mercaptoethanol, dithiothreitol, and dithioerythritol), mercaptoacetic acid, sodium thioglycolate, thiolactic acid, thioglycoamide, glycerol monothioglycolate, borohydrides (e.g., sodium borohydride), ternary amines, thiocyanates such as sodium thiocyanate, thiosulfates such as sodium thiosulfate, cyanides such as sodium cyanide, thiophosphates such as sodium thiophosphate, arsenites such as sodium arsenite, phosphines such as triphenyl phosphine, phenols such as thiophenol and p-nitrophenol, betaines, lithium aluminum hydride, guanidine hydrochloride, stannous chloride, hydroxylamine, LiHB (C₂H₅)₃, zinc metal, Raney nickel, hydrazines, and substituted hydrazines. Two or more of the reducing agents can also be used in combination to reduce the viscosity of the exudate.

Other suitable reducing agents include stabilized radicals. Examples of stabilized radicals that can act as reducing agents include alkyl tin hydrides, such as tributyl tin hydride, organic peroxides, such as benzoyl peroxide and di-tert-butyl peroxide, azobisisobutyronitrile (AIBN), and triphenyl carbenium-salts.

In another embodiment, the bodily exudate modifying agents are agents that can activate enzymatic autolysins, such as peptidoglycan hydrolases, in the bacteria present in bodily exudates. These activated autolysins will catalyze the lysis of the bacterial cell wall through the use of the cell's own enzymes. As the bacterial cell wall is destroyed, water is liberated from inside the bacterial cell. This released water from inside of the cell produces a dilution effect around the exudate, which results in the reduction of the viscosity of the bodily exudate.

One suitable example of a bodily exudate modifying agent that can activate autolysins in bacteria found in exudate is a metal-based modifying agent. As used herein, the term “metal-based modifying agent” refers to any chemical compound containing a metal capable of activating an autolysin, which can reduce the viscosity of bodily exudates. In one embodiment, the metal-based modifying agents include, but are not limited to, magnesium-based modifying agents, barium-based modifying agents, calcium-based modifying agents, and combinations thereof.

Suitable magnesium-based modifying agents are magnesium oxide, magnesium hydroxide, and magnesium chloride. Suitable barium-based modifying agents are barium oxide, barium hydroxide, and barium chloride.

Specifically, calcium-based modifying agents are required for activating calpain-type protease autolysins. Some suitable calcium-based modifying agents useful for the present invention can include calcium oxide, calcium hydroxide, calcium chloride, and calcium carbonate.

The metal-based modifying agents can also suitably be metal salts. Suitably, the metal salt can be selected from the group consisting of iron salts, aluminum salts, calcium salts, and combinations thereof.

Additionally, various peptides can be used to activate autolysins in bacteria and reduce the viscosity of the exudate. Suitably peptides for use in the present invention are cationic peptides. Cationic peptides cause a deregulation of the anionic and amphiphilic regulators of the autolytic wall components, such as enzymes (muramidases), lipoteichoic acid, and Forssman antigens. This deregulation results in hydrolysis of the peptidoglycan found in the normal bacteria cell wall, bacteriolysis, and cell death. For example, a suitable cationic peptide is niacin.

Some pore-forming toxins can further induce autolysins. These toxins shut down transport channels in bacterial cell walls, forcing the channels to remain open. As such, water is released into the absorbent article from inside the bacterial cell. As stated above, this produces a dilution effect, which reduces the viscosity of bodily exudates. Suitably pore-forming toxins include alpha-toxins, cytolysin A, and seticholysins.

In addition to agents that can activate autolysins, certain nanoemulsions, alternatively known as nanoparticles, can cause lysis of bacterial cell walls. The bacterial cell wall ruptures when the walls come into contact with the nanoemulsions. As stated above, this results in the release of water from inside the bacterial cell, causing a dilution effect, which reduces the viscosity of the bodily exudates.

Methods for preparing nanoemulsions or nanoparticles suitable for use in the present invention are well known and disclosed, for example, in U.S. Pat. Nos. 6,558,941 and 6,623,761. For example, in one embodiment, nanoemulsions can be prepared through the process of wet grinding. Wet grinding involves the mechanical crushing of brittle particles, using hard beads made of glass, porcelain, zirconium oxide, or similar materials of about 1-2 mm in diameter, and aqueous solution of a hydrophilic material. The hydrophilic solution, which can be a surface active agent, surface modifier, or surface stabilizer, prevents aggregation or caking of the ground particles.

One embodiment suitable for the present invention includes nanoemulsions composed of oil particles, the surfaces of which are occupied by an amphoteric emulsifier in aqueous dispersions. These oil particles are of a diameter of less than about 100 nm, and more suitably, of less than 40 nm. Other suitable nanoemulsions for use in the present invention may include vegetable oil in water emulsions, triglycerides in water emulsions, fatty acid esters in water emulsions, and combinations thereof.

In another embodiment, surfactants, and specifically mild-type surfactants, can be used as bodily exudate modifying agents. Surfactants lower the surface tension of liquids, and as such, the use of mild surfactants in the present invention will facilitate the release of moisture, thus enhancing the breakdown of the bodily exudates. Mild surfactants are typically preferred to reduce the likelihood of the surfactant damaging the skin. Suitable mild surfactants include sodium mono lauryl phosphate, potassium mono lauryl phosphate, diethanolamine mono lauryl phosphate, triethanolamine mono lauryl phosphate, sodium mono coco phosphate, potassium mono coco phosphate, triethanolamine mono coco phosphate, sodium mono capric phosphate, potassium mono capric phosphate, triethanolamine mono capric phosphate, non-ionic surfactants, such as PLURONIC surfactants, and combinations thereof.

While the use of the above discussed bodily exudate modifying agents will generally result in a reduced viscosity of the bodily exudate upon contact therewith, some of the bodily exudate modifying agents, such as lipase and protease enzymes, may cause an irritation on the skin surface that is uncomfortable and can predispose the skin to infection by microorganisms if they remain in contact with the skin for any appreciable period of time. As such, the cleansing systems of the present invention can optionally further include a bodily exudates modifying agent neutralizer. As used herein, the term “bodily exudate modifying agent neutralizer” includes any chemical compound that can chemically neutralize or inhibit the effect of the bodily exudate modifying agents and reduce their potentially harmful effects on the skin surface.

The products used in the cleansing systems can suitably include a bodily exudate modifying agent neutralizer in an amount of from about 0.01% (by weight of the product) to about 10% (by weight of the product). More suitably, the products of the cleansing system of the present invention include bodily exudate modifying agent neutralizers in an amount of from about 0.1% (by weight of the product) to about 0.5% (by weight of the product).

Typically, the anti-adherent formulation of the present invention can act as a bodily exudates modifying agent neutralizer. Preferably, however, additional bodily exudate modifying agent neutralizers may be used. In one embodiment, the bodily exudate modifying agent neutralizer is an enzyme inhibitor. Inhibitors of enzyme activity are well known and are typically classified as competitive inhibitors, which compete with the substrate for binding at the active site on the enzyme, and non-competitive inhibitors, which bind to a site other than the active site to inactivate the enzyme. Suitably, enzyme inhibitors useful in the skin products described herein include protease inhibitors, lipase inhibitors, elastase inhibitors, urease inhibitors, amylase inhibitors, and combinations thereof. More suitably, the enzyme inhibitors are selected from the group consisting of soybean trypsin inhibitor, lima bean protease inhibitor, corn protease inhibitor, Bowman-Birk inhibitor, pancreatic trypsin inhibitor, ovomucoids, chymostatin, leupeptin and its analogs, bestatin and its analogs, antipain, antithrombin III, hirudin, cystatin, α₂-macroglobulin, α₁-antitrypsin, pepstatin and its analogs, TLCK, TPCK, tranexamic acid and its salts, glycyrrhizic acid and its salts, stearylglycyrrhetinate, 18-β-glycyrrhetinic acid and its salts, colloidal oat extracts, elhibin, 4-(2-aminoethyl)-benzenesulfonylfluoride HCl, quercetin, phytic acid and its salts, ethylenediamine tetraacetic acid (EDTA) and its salts, hexamidine and its salts, pentamidine and its salts, benzamidine and its salts and derivatives, p-aminobenzamidine and its salts and derivatives, guanidinobenzoic acid and its salts and derivatives, alkyl hydroxamic acid and its salts and derivatives, phosporamidate and its derivatives, water soluble salts of metals, zinc salts of both saturated and unsaturated monocarboxylic acids, glycerol triesters of fatty acids, block copolymers of propylene oxide and ethylene oxide, chlorhexidine, cholestyramine, acarbose, voglibose, miglitol, emiglitate, camiglibose, pradimicin Q, salbostatin, tendamistat, trestatins, inhibitors derived from plants, such as from wheat, rice, maize, barley, and other cereal grains, beans, and seaweed, tetrahydrolipstatin, lipstatin, valilactone, esterastin, ebelactone A and B, 1,6-di(O-(carbamoyl)cyclohexanone oxime)hexane, and combinations thereof.

In another embodiment, the bodily exudate modifying agent neutralizer is a skin irritant sequestering agent. As used herein, the term “sequestering agent” means a material that can adsorb a target molecule, such as a fecal protease, by covalent or non-covalent mechanisms. In certain preferred embodiments, the affinity for the irritant is high, rapid, and irreversible. Adsorption of the irritant to the sequestering agent should preclude or significantly diminish the ability of a target irritant to penetrate into, and potentially through, the stratum corneum. As used herein, the term “sequestration” is defined as the process of binding of an irritant to a sequestering agent, by covalent or non-covalent mechanisms.

The adsorption of these target molecules, i.e., the bodily exudate modifying agents, minimizes their ability to penetrate into the skin and cause skin irritation. Suitable skin irritant sequestering agents can include clays. Particularly, the clay is suitably selected from the group consisting of bentonite, laponite, montmorillonite, beidelite, hectorite, saponite, stevensite, and combinations thereof. Also suitable as sequestering agents are silica, titanium dioxide, hydroxyapatite, alumina, ion-exchange resin, and combinations thereof.

In another embodiment, the bodily exudate modifying agent neutralizer is an oxidizing agent. In a reduction-oxidation reaction, the oxidizing agent oxidizes or extracts electrons from the reducing agent. As such, the effects of bodily exudate modifying agents like reducing agents will be neutralized by the oxidizing agent. Suitable oxidizing agents are citric acid, malic acid, alphahydroxy acid, hydrogen peroxide, and peroxide.

In another embodiment, the bodily exudate modifying agent neutralizer is a binding protein. A binding protein, like the enzyme inhibitors discussed above, will bind to an active or inactive site on the bodily exudate modifying agent, inhibiting the action of the bodily exudate modifying agent. Suitable binding proteins for the present invention include serum albumin, histone proteins, plant proteins, animal proteins, fish proteins, yeast extract, algal proteins, and bacterial proteins.

In another embodiment, the bodily exudate modifying agent neutralizer is a zwitterion. Zwitterions, which carry both a negative and a positive charge, can act as an acid or base. As such, zwitterions for the use in the present invention can neutralize the bodily exudate modifying agents discussed above, particularly when the agents are mild surfactants, by neutralizing the charge of the modifying agent. Suitable zwitterions include, for example, amino acids such as alanine and betaine glycine.

In one embodiment of the present invention, the anti-adherent formulation, the cationic compound, and/or the bodily exudate modifying agent, or one or more components of these formulations or compounds, may be encapsulated in a shell material prior to being introduced into or onto the product. When the product is wiped or rubbed across the skin, the capsules break open due to the shear of the wiping and release the formulation or component(s). Additionally, the product may be dispensed from a dispensing unit that, upon dispensing, creates shear and causes the capsules to break and release the formulation or component(s). Suitable microencapsulation shell materials include cellulose-based polymeric materials (e.g., ethyl cellulose), carbohydrate-based materials (e.g., cationic starches and sugars) and materials derived therefrom (e.g., dextrins and cyclodextrins) as well as other materials compatible with human tissues.

The microencapsulation shell thickness may vary depending upon the formulation utilized, and is generally manufactured to allow the encapsulated formulation or component to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should also be constructed such that humidity from atmospheric conditions during storage, shipment, or wear will not cause a breakdown of the microencapsulation layer and result in a release of the formulation or component.

Microencapsulated formulations or components applied directly to the product should be of a size such that the user cannot feel the encapsulated shell on the skin during use. Typically, the capsules have a diameter of no more than about 25 micrometers, and desirably no more than about 10 micrometers. At these sizes, there is no “gritty” or “scratchy” feeling on the skin when the product is utilized.

When the products of the present invention comprise base substrates, the anti-adherent formulations, cationic compounds, and/or bodily exudate modifying agents described herein can be introduced onto the suitable base substrates utilizing various techniques known in the art. For example, the formulations or compounds may include a suspending or thickening agent to suspend the formulation or compound such that it can be gravure or flexographically coated, sprayed, ink-jet printed, or slot coated onto the base substrate in the desired amount. Suitable thickening agents may include, for example, clays, cellulose derivatives such as carboxymethyl cellulose and carboxypropyl cellulose, natural gums such as guar gum and xanthan gum, and acrylate polymers.

As will be recognized by one skilled in the art based on the disclosure herein, the products comprising a base substrate described herein can be manufactured and sold to consumers in various product forms. For example, the products could be manufactured and sold in roll form, as individual sheets, or in stacks of individual sheets. In any of these forms, the product can be in wet form similar to a wet wipe, or could be dry to the touch such that a consumer would wet the product prior to use.

In addition to the components of the various formulations and compounds described herein, the products may additionally comprise one or more optional components to impart additional benefits to the cleansing systems of the present invention. Suitable optional components include, for example, skin protectants, powders, anti-biotics, anti-microbials, anti-inflammatories, odor control agents, fragrances, colorants, vitamin E, aloe extract, and preservatives.

In view of the above, it will be seen that the several objects of the invention are achieved and other advantageous results obtained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. A cleansing system for improving skin health, the system comprising:
a first product comprising an anti-adherent formulation; and
a second product comprising a cationic compound capable of binding contaminants located on or near the skin, wherein the cationic compound has an effective charge density of from about 0.1 microequivalents/g to about 8000 microequivalents/g, and wherein the second product has an effective charge density of at least about 2000 microequivalents/100 g.

2. The cleansing system as set forth in claim 1 wherein the anti-adherent formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

3. The cleansing system as set forth in claim 2 wherein the emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

4. The cleansing system as set forth in claim 2 wherein the structuring agent is selected from the group consisting of animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

5. The cleansing system as set forth in claim 2 wherein the rheology modifier is selected from the group consisting of combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, and silicas.

6. The cleansing system as set forth in claim 2 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

7. The cleansing system as set forth in claim 1 wherein the first product is selected from the group consisting of ointments, lotions, creams, salves, aerosols, gels, foams, washes, mists, and sprays.

8. The cleansing system as set forth in claim 7 wherein the first product comprises from about 0.01% (by weight of the product) to about 5% (by weight of the product) of the anti-adherent formulation.

9. The cleansing system as set forth in claim 1 wherein the first product further comprises a base substrate.

10. The cleansing system as set forth in claim 9 wherein the first product comprises from about 1% (by weight of the base substrate) to about 25% (by weight of the base substrate) of the anti-adherent formulation.

11. The cleansing system as set forth in claim 1 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

12. The cleansing system as set forth in claim 1 wherein the second product further comprises a base substrate.

13. The cleansing system as set forth in claim 12 wherein the second product is selected from the group consisting of diapers, training pants, adult incontinence garments and pads, feminine napkins, hand towels, and wound dressings.

14. The cleansing system as set forth in claim 12 wherein the second product comprises from about 0.1% (by weight of base substrate) to about 10% (by weight of base substrate) of the cationic compound.

15. A product for improving skin health comprising a base substrate, an anti-adherent formulation and a cationic compound capable of binding contaminants located on or near the skin, wherein the cationic compound has an effective charge density of from about 0.1 microequivalents/g to about 8000 microequivalents/g, and wherein the product has an effective charge density of at least about 2000 microequivalents/100 g.

16. The product as set forth in claim 15, wherein the anti-adherent formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

17. The product as set forth in claim 16 wherein the emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

18. The product as set forth in claim 16 wherein the structuring agent is selected from the group consisting of animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

19. The product as set forth in claim 16 wherein the rheology modifier is selected from the group consisting of combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, and silicas.

20. The product as set forth in claim 16 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

21. The product as set forth in claim 15 wherein the product comprises from about 0.01% (by weight of the base substrate) to about 5% (by weight of the base substrate) of the anti-adherent formulation.

22. The product as set forth in claim 15 wherein the cationic compound is selected from the group consisting of quaternary compounds, polyelectrolytes, octadecyldimethoxylsilpropylammonium chloride, 1-methyl-2Noroleyl-3-oleyl-amidoethyl imidazoline methylsulfate, and 1-ethyl-2-Noroleyl-3-oleyl-amidoethyl imidazoline ethylsulfate.

23. The product as set forth in claim 15 wherein the base substrate comprises from about 0.1% (by weight of the base substrate) to about 10% (by weight of the base substrate) of a cationic compound.

24. The product as set forth in claim 15 selected from the group consisting of diapers, training pants, adult incontinence garments and pads, feminine napkins, hand towels, and wound dressings.

25. A cleansing system for improving skin health, the system comprising:
a first product comprising an anti-adherent formulation; and
a second product comprising a base substrate, a bodily exudate modifying agent, and a bodily exudates modifying agent neutralizer, the bodily exudate modifying agent being capable of reducing the viscosity of bodily exudates upon contact therewith.

26. A process for improving skin cleansing, the process comprising:
contacting the skin with a first product comprising an anti-adherent formulation; and
contacting the skin with a second product comprising a cationic compound capable of binding contaminants located on or near the skin, wherein the cationic compound has an effective charge density of from about 0.1 microequivalents/g to about 8000 microequivalents/g, and wherein the second product has an effective charge density of at least about 2000 microequivalents/100 g.

27. The process as set forth in claim 26 wherein the anti-adherent formulation comprises from about 30% (by weight) to about 88.99% (by weight) emollient, from about 10% (by weight) to about 68% (by weight) structuring agent, from about 1% (by weight) to about 25% (by weight) rheology modifier, and from about 0.01% (by weight) to about 1% (by weight) anti-adherent compound.

28. The process as set forth in claim 27 wherein the emollient is selected from the group consisting of petrolatum, mineral oil, mineral jelly, isoparaffins, vegetable oils, avocado oil, borage oil, canola oil, castor oil, chamomile, coconut oil, corn oil, cottonseed oil, evening primrose oil, safflower oil, sunflower oil, soybean oil, sweet almond, lanolin, partially hydrogenated vegetable oils, sterols and sterol derivatives, polydimethylsiloxanes, methicone, cyclomethicone, dimethicone, dimethiconol, trimethicone, organo-siloxanes, silicone elastomer, gums, resins, fatty acid esters (esters of C₆-C₂₈ fatty acids and C₆-C₂₈ fatty alcohols), glyceryl esters and derivatives, fatty acid ester ethoxylates, alkyl ethoxylates, C₁₂-C₂₈ fatty alcohols, C₁₂-C₂₈ fatty acids, C₁₂-C₂₈ fatty alcohol ethers, Guerbet alcohols, Guerbet Acids, Guerbet Esters, and combinations thereof.

29. The process as set forth in claim 27 wherein the structuring agent is selected from the group consisting of animal waxes, vegetable waxes, mineral waxes, synthetic waxes, polymers, bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba buffer, jojoba esters, jojoba wax, lanolin wax, microcrystalline wax, mink wax, motan acid wax, motan wax, ouricury wax, ozokerite parrafin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers, C₁₈-C₄₅ olefins, poly alpha olefins, hydrogenated vegetable oils, polyhydroxy fatty acid esters, polyhydroxy fatty acid amides, ethoxylated fatty alcohols and esters of C₁₂-C₂₈ fatty acids, and esters of C₁₂-C₂₈ fatty alcohols, and combinations thereof.

30. The process as set forth in claim 27 wherein the rheology modifier is selected from the group consisting of combinations of alpha-olefins and styrene or polyethylene alone or in combination with mineral oil or petrolatum, di-functional alpha-olefins and styrene alone or in combination with mineral oil or petrolatum, combinations of alpha olefins and isobutene, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, styrene-ethylene/propylene copolymers, and silicas.

31. The process as set forth in claim 27 wherein the anti-adherent compound is selected from the group consisting of alginic acid, beta-benzal-butyric acid, botanicals, casein, dextrans, farnesol, flavones, fucans, galactolipid, kininogen, hyaluronate, inulin, iridoid glycosides, nanoparticles, perlecan, phosphorothioate oligodeoxynucleotides, pluronic surfactants, poloxamer 407, polymethylmethacrylate, silicone, sulphated exopolysaccharides, tetrachlorodecaoxide, and combinations thereof.

32. The process as set forth in claim 26 wherein the first product is selected from the group consisting of ointments, lotions, creams, salves, aerosols, gels, foams, washes, mists, and sprays.

33. The process as set forth in claim 32 wherein the first product comprises from about 0.01% (by weight of the product) to about 5% (by weight of the product) of the anti-adherent formulation.

34. The process as set forth in claim 26 wherein the first product further comprises a base substrate.

35. The process as set forth in claim 34 wherein the first product comprises from about 1% (by weight of the base substrate) to about 25% (by weight of the base substrate) of the anti-adherent formulation.

36. The process as set forth in claim 26 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

37. The process as set forth in claim 26 wherein the second product further comprises a base substrate.

38. The process as set forth in claim 37 wherein the second product is selected from the group consisting of diapers, training pants, adult incontinence garments and pads, feminine napkins, hand towels, and wound dressings.

39. The process as set forth in claim 37 wherein the second product comprises from 0.1% (by weight of the base substrate) to about 10% (by weight of the base substrate) of a cationic compound.
